(19) United States
(12) Patent Application Publication
(10) Pub. No.: US 2003/0019120 A1
(43) Pub. Date: Jan. 30, 2003

(54) Fingernail measuring method and apparatus

(76) Inventor: Aida Davenport, Modesto, CA (US)

Correspondence Address: MARK D MILLER, KIMBLE, MACMICHAEL & UPTON, 5260 NORTH PALM AVENUE, SUITE 221, FRESNO, CA, 93704, US

(21) Appl. No.: 09/919,984

(22) Filed: Jul. 30, 2001

(51) Int. Cl. A61B 5/103

(52) U.S. Cl. 33/512

(57) ABSTRACT

The present invention is a cap bearing measurement indicia that may be used independently, in conjunction with, or integrated into a fingernail file or other instrument to determine the length of a fingernail. The invention is designed to allow the user to measure the length of the fingernails, and trim the fingernails to desired lengths according to the measurements taken.

### BACKGROUND OF THE INVENTION

1. Field of the Invention

The present invention relates generally to fingernail care, and more particularly to a unique finger nail measuring device that may be is incorporated into a finger nail file for use in achieving finger nails of consistent length.

2. Description of the Prior Art

Fingernail files, fingernail clippers and related products are well known in the art. Over time, it has become fashionable to extend the length of one's fingernails by various natural and artificial means. Files and clippers are used to establish the length of one's fingernails, however it has always been difficult to obtain the same or consistent lengths for all ten fingernails of both hands. It has also been particularly difficult to maintain elongated fingernail lengths over time, as the natural fingernails grow, sometimes unevenly, and as the fingernails are subjected to chipping, breaking and cracking during ordinary use.

U.S. Pat. No. 5,070,392 describes an artificial fingernail tip having a pattern thereon used for cutting the artificial nail to a desired length prior to attachment. U.S. Pat. No. 6,012,461 describes a teaching system for use in creating artificial fingernails. Neither of these inventions involves measuring or determining the length of the fingernail after it has been attached to the natural nail, but instead establish fingernail lengths prior to attachment. U.S. Pat. No. 5,832,936 describes an artificial fingernail tip having length indicating indicia located thereon for use in establishing the length of the nail, the indicia being buffed out after attachment. However, once the indicia is buffed out (and painted over) neither this invention nor either of the previous inventions allows for continued monitoring and maintenance of the now elongated fingernails over time as the natural fingernails grow out, and as unavoidable chipping and breaking occurs. None of these inventions allows for measuring or monitoring the length of naturally grown (non-artificial) elongated fingernails.

It is therefore desirable to provide a way to accurately measure the length of an existing elongated fingernail, whether natural or artificial, in order to maintain consistent lengths of all fingernails of both hands.

### SUMMARY OF THE INVENTION

The present invention provides a cap bearing measurement indicia that may be used independently or in conjunction with a fingernail file or other instrument to determine the length of a fingernail. The cap is designed to allow the user to measure the length of the fingernails, and trim the fingernails to desired lengths according to the measurements taken. The cap may be made of rubber, plastic or other suitable material and attached to the end of a fingernail file, cuticle rod, pencil, or other elongated instrument. Alternatively, the cap may be integrated into one end of a nail file or other instrument and used a single unit.

The cap of the present invention has a pointed configuration with measuring indicia across an outer surface, the measurements beginning at the pointed tip of the cap and increasing as the distance from the tip increases. The measurements extend across the surface of the cap, and may be in any desired increments (e.g. millimeters, sixteenths of an inch, etc.) according to the needs of the user.

In use, the tip of the cap is inserted underneath an installed or naturally grown fingernail with the tip touching the end of the finger underneath the nail. The measurement indicia is below the fingernail, and the portion protruding out from underneath the fingernail discloses the length of the fingernail from the end of the finger to the tip of the nail. This measuring process is repeated for all fingers. A desired length (or lengths, if different fingers/thumbs are to have different nail lengths) is then determined. Thereafter, the fingernails are trimmed to the desired length(s), using the present invention to measure and check the lengths as the trimming process takes place.

Incorporating the measuring cap of the present invention into a nail file facilitates easy checking of the results of filing by allowing the filing and measuring operations to be performed by the same instrument.

It is therefore a primary object of the present invention to provide a device for measuring the lengths of previously installed or naturally grown fingernails in order to facilitate trimming the fingernails to desired and consistent lengths.

It is also a primary object of the present invention to provide an integrated nail file and measuring device for measuring and trimming artificial or naturally grown fingernails to desired lengths.

It is another object of the present invention to provide a stand alone fingernail measuring device that may be attached to a nail file, pencil, cuticle rod, or other instrument.

Additional objects of the invention will be apparent from the detailed descriptions and the claims herein.

### BRIEF DESCRIPTION OF THE DRAWINGS

FIG. 1 is a front view of the cap of the present invention attached to a nail file.

FIG. 2 is an enlarged view of the attached cap of FIG. 1.

FIG. 3 is a perspective view of the present invention in use measuring a fingernail.

FIG. 4 is a side view of the invention of FIG. 1.

FIG. 5 is a front view of the measuring device of the present invention integrated into a nail file as a single instrument.

### DETAILED DESCRIPTION OF THE PREFERRED EMBODIMENTS

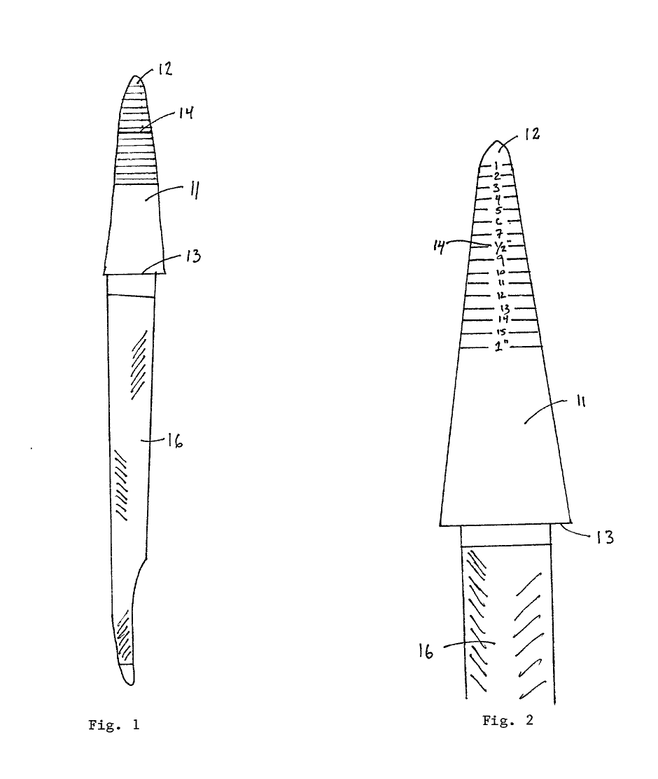

Referring to the drawings wherein like reference characters designate like or corresponding parts throughout the several views, and referring particularly to FIGS. 1 and 2, it is seen that the invention includes a cap 11 having a pointed end 12 and a wider opposite end 13. Measurement indicia 14 are provided beginning at the pointed end 12 and increasing towards wider end 13. Cap 11 may be provided as a stand-alone device, or detachably attached to a nail file 16 or other suitable instrument as shown in FIGS. 1-3.

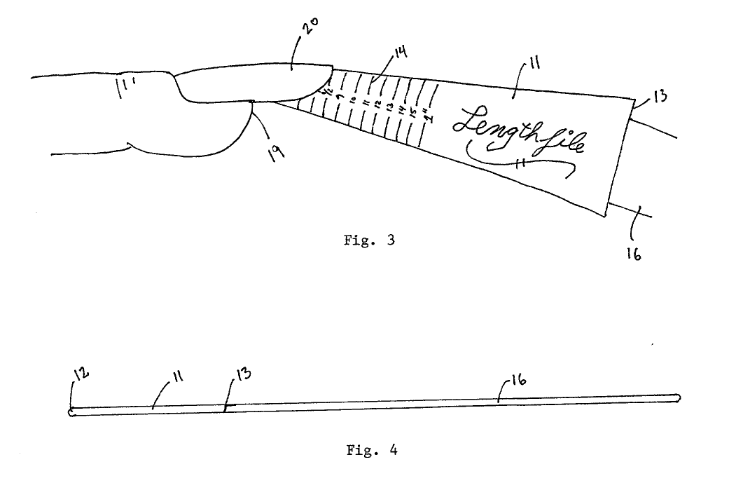

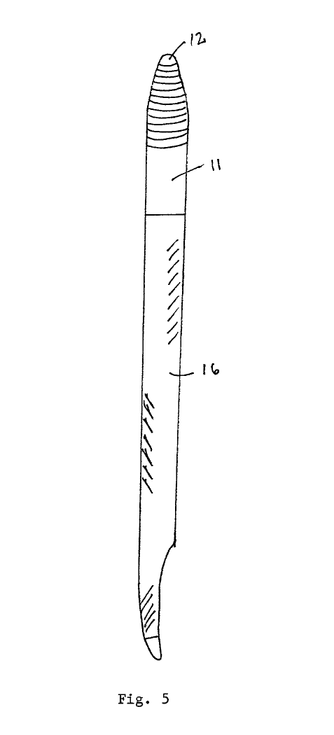

The length indicating indicia 14 are illustrated in sixteenths of an inch, sequentially arranged from 0 to 16, and beyond, if desired, indicating 1/16″ graduations. The measurement lines should cover the entire flat surface of the cap so that they may be seen extending out from under the fingernail (see FIG. 3). The indicia lines may be straight, but are preferably curved in order to more closely conform to the rounded shape of the fingernail. It is to be appreciated that other numbers or other length indicating indicia may be used, for example, an arrangement of letters. Similarly, the graduations may be in something other than 1/16″ increments and in a different order. Moreover, the numbers may be painted, etched, embossed or printed onto the unit. Advertising, identification or other information may also be provided in the space beyond the last measurement increment.

In use, the cap 11 of the present invention is inserted underneath a fingernail 20 as shown in FIG. 3. In the illustration, the fingernail extends approximately one-half inch (½) from the end of the finger 19. This measurement is then used to determine how much nail 20 is to be trimmed in order to have a desired consistent length. It is to be appreciated that the user may desire different fingernails to have different lengths (e.g. index fingernails to be longer than all others; thumb nails to be shorter than all others, etc.).

It is to be understood that variations and modifications of the present invention may be made without departing from the scope thereof. It is also to be understood that the present invention is not to be limited by the specific embodiments disclosed herein, but only in accordance with the appended claims when read in light of the foregoing specification.

What is claimed is:

1. A fingernail measuring device comprising a detachable pointed cap having measurement indicia at the pointed end thereof, said indicia increasing with distance from said pointed end.

2. The measuring device of claim 1 wherein said pointed cap is flat and has a front and rear surface, and said measurement indicia includes a plurality of equidistantly spaced arcuate lines extending completely across one of said surfaces.

3. The measuring device of claim 2 wherein said arcuate lines extend completely across both of said surfaces.

4. The measuring device of claim 2 wherein said device is detachably attached to a fingernail file.

5. The measuring device of claim 2 wherein said device is detachably attached to a cuticle rod file.

6. An instrument for measuring and modifying the length of a fingernail comprising an fingernail file having at least one pointed end with measurement indicia integrated into said pointed end.

7. The instrument of claim 6 wherein said pointed end is flat and has a front and rear surface, and said measurement indicia includes a plurality of equidistantly spaced arcuate lines extending completely across one of said surfaces.

8. The instrument claim 7 wherein said arcuate lines extend completely across both of said surfaces.

9. A method for obtaining a desired length of an installed elongated fingernail comprising the steps of:
a. inserting a device having a pointed end with measurement indicia located thereon underneath said fingernail such that the point touches the end of the finger;
b. determining the desired length for the fingernail according to the measurement indicia; and
c. trimming said fingernail to the desired length.

10. The method of claim 9 including the additional step of repeating steps a-c for each fingernail of each hand.

11. The method of claim 9 wherein said device comprises a fingernail file having at least one pointed end with measurement indicia integrated into said pointed end.

12. The instrument of claim 11 wherein said pointed end is flat and has a front and rear surface, and said measurement indicia includes a plurality of equidistantly spaced arcuate lines extending completely across one of said surfaces.

13. The instrument claim 12 wherein said arcuate lines extend completely across both of said surfaces.
